(19) United States
(12) Patent Application Publication
(10) Pub. No.: US 2004/0223983 A1
(43) Pub. Date: Nov. 11, 2004

(54) Pharmaceutical preparations, use of these preparations and process for increasing the biovailability of pharmaceutical substances to be administered perorally

(76) Inventors: Philip Lienau, Berlin (DE); Thomas Backensfeld, Berlin (DE); Andreas Reichel, Berlin (DE); Tanja Jung, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/679,507

(22) Filed: Oct. 7, 2003

(30) Foreign Application Priority Data
Oct. 8, 2002 (DE) 102 47 399.4

(51) Int. Cl. A61K 9/00

(52) U.S. Cl. 424/400

(57) ABSTRACT

The invention relates to pharmaceutical preparations that contain at least one emulsifier, at least one auxiliary emulsifier and/or solvent as well as at least one lipid, characterized in that the mass ratio of emulsifier to auxiliary emulsifier and/or solvent (Smix) is 1:1 to 9:1 and the total lipid proportion is >0% (m/m), whereby this preparation at least partially inhibits at least one intestinal enzyme and/or at least one intestinal efflux system. These preparations can be used to increase the bioavailability of pharmaceutical substances that are lipophilic and/or substrates of intestinal metabolizing enzymes and/or intestinal efflux systems, especially steroids.

The invention relates to a pharmaceutical preparation that contains at least one emulsifier, at least one auxiliary emulsifier and/or solvent and at least one lipid, as well as the use of the preparation according to the invention as a peroral pharmaceutical agent and a process for increasing the bioavailability of pharmaceutical substances to be administered perorally.

The following technical terms are used for the purposes of this invention according to the definitions below.

For the purposes of this invention, the term pharmaceutical substance is used synonymously to active ingredient and/or pharmaceutical agent and includes substances that have a main pharmacological action as well as those that do not have any main pharmacological action.

For the purposes of this invention, the term pharmaceutical agent is used synonymously to medication and means that a pharmaceutical preparation that contains at least one pharmaceutical substance is used for therapeutic, prophylactic and/or diagnostic purposes.

For the purposes of this invention, dosage form means the form that is to be administered, i.e., the dispensing form of the pharmaceutical agent, whereby those dispensing forms that must still be converted before use into the actual dispensing form also fall under this.

For the purposes of this invention, pharmaceutical preparation is used synonymously to basic mixture, preconcentrate, formulation or active ingredient carrier system and contains pharmaceutical adjuvants that can control or support the pharmaceutical substance action, as well as possibly other basic substances that are required for a production of dosage forms, i.e., dispensing forms of preparations of pharmaceutical substances.

For the purposes of this invention, emulsifiers, auxiliary emulsifiers, solvents and lipids are pharmaceutical adjuvants, whereby emulsifiers and auxiliary emulsifiers belong to the group of surfactants, i.e., they have a hydrophilic and a lipophilic portion in the molecule. For the purposes of this invention, emulsifiers are hydrophilic in their entirety, i.e., they have an HLB (hydrophile-lipophile balance) value of >10; optimally >12. Auxiliary emulsifiers that are used for the purposes of the invention are lipophilic in their entirety, i.e., they have an HLB value of <10; optimally <8. For the purposes of the application, solvents have the object to improve the solubility and the self-emulsifiability of the existing phases of a formulation according to the invention. In particular, organic solvents, advantageously alcohols, polyethylene oxide glycols (PEG) and modified PEG, e.g., etherified PEG (Transcutol®P) are considered.

For the purposes of the application, Smix (surfactant mixture) reflects the mass ratio of emulsifier to auxiliary emulsifier.

For the purposes of this invention, m=mass and v=volume in the data (v/v+m), (m/v) or (m/m).

For the purposes of this invention, substrate means at least one intestinal enzyme and/or an intestinal efflux system, such that substances, in particular pharmaceutical substances in the intestine, interact with the respective enzyme or efflux systems so that they are metabolized and/or actively flushed from the intestinal epithelium into the lumen of the bowels.

For the purposes of this application, the terms intestinal enzyme and/or intestinal efflux system stand for enzymes/efflux systems that occur, i.a., in intestinal tissue and at least partially prevent there the absorption of pharmaceutical active ingredients and thus can reduce bioavailability after peroral administration.

For the purposes of this invention, inhibiting means that in the presence of the preparation or the adjuvants according to the invention that are used for the preparation according to the invention, substrates of intestinal enzymes or intestinal efflux systems are metabolized by the latter to a lesser extent and/or are actively flushed from cells by efflux systems.

For the purposes of this invention, bioavailability of pharmaceutical substances is defined as the total portion of pharmaceutical substance that is systemically available in terms of time.

The potential usefulness of pharmaceutical substances depends on, i.a., the bioavailability such that in the pharmaceutical development, a special advantage exists in optimizing the bioavailability of pharmaceutical substances. The bioavailability that is referenced for the purposes of this invention is the rate and/or the extent in which the therapeutically, prophylactically or diagnostically active portion of a pharmaceutical agent is released from a dosage form and resorbed or is available at the site of action. The bioavailability is measured via the concentration of the respective pharmaceutical substance or its metabolites in bodily fluids, such as, e.g., blood, based on time.

The bioavailability of pharmaceutical substances that are resorbed in particular via the gastrointestinal tract depends on the solubility and thus the resorbability of the pharmaceutical substances. Thus, in the prior art, the solubility of especially strong lipophilic and/or poorly water-soluble pharmaceutical substances that consequently have a slow and incomplete release of pharmaceutical substances and/or resorption, e.g., is increased by lipid-based formulations that promote a formation of dissolved phases and thus an increase in bioavailability of these pharmaceutical substances.

In U.S. Pat. No. 5,391,377 A, a two-phase pharmaceutical composition is disclosed that consists of a long-lasting release component, essentially C₁₂-C₂₄-fatty acids, and a pharmaceutically active substance, preferably lipophilic pharmaceutical substances, as well as a non-long-lasting release component, a C₁₂-C₂₄-fatty acid.

In WO 94/09788 A1, a pharmaceutical preparation is disclosed that improves the solubility of HIV-protease inhibitors by organic solvents, especially alcohols, and optionally in addition by an acid.

In U.S. Pat. No. 5,342,625 A, a cyclosporin-containing microemulsion preconcentrate and microemulsion is disclosed that produces an oil-in-water emulsion (O/W emulsion) and is to result in a higher bioavailability with lower inter-individual and intra-individual ranges of scatter of the resorption level.

In EP 670715 B1, an anhydrous pharmaceutical preparation that consists of a surfactant, a co-surfactant and a liphophilic phase was disclosed as a solution of the problem. These preparations have transparent, single-phase areas in a pseudotemary phase diagram (emulsifier, lipid, water) when water is added in portions. It is disadvantageous in this case that the transparent, single-phase, water-dilutable area is limited to a water portion of at most 70% so that, moreover, in the so-called exhaust percentage, cloudiness of the system, in particular precipitation or crystallization of water-insoluble pharmaceutical substances and thus worsened resorption conditions of the pharmaceutical substances prevail.

In Lienau et al., Proc. EUFEPS World Conference on Drug Absorption and Drug Delivery, Copenhagen, Jun. 18-20, 2001, p. 106, and Lienau et al., Proc. 4^{th} World Meeting ADRITELF/APGI/APV, Florence, Apr. 8/11, 2002, p. 1463 f, pharmaceutical preparations are described that have an emulsifier, auxiliary emulsifier and a lipid and, relative to the preparations, have the advantage according to EP 670715 B1 that they have a transparent, single-phase area over 70% (m/m) of the water portion in the case of water dilution by steps in a pseudoternary phase diagram.

A drawback of the previously described documents consists in that these preparations teach only an increase in bioavailability of strongly lipophilic and/or water-insoluble pharmaceutical substances via the increase in solubility, but an additional metabolic reduction of the bioavailability of in particular also hydrophilic pharmaceutical substances is not achieved.

The bioavailability of pharmaceutical substances that are to be administered perorally is metabolized in particular in the gastrointestinal tract by enzymes of the first phase, for example from the superfamily of the cytochrome P450 monooxygenases, in particular CYP-3A or the 17β-hydroxy-steroid hydrogenases (17β-HSD, cf. in: SANO, T., et al., Clin. Sci. 2001, 101 (5): 485-491), in particular the 17β-HSD 2 isoform, and the second phase, for example sulfatases. In addition, the bioavailability of pharmaceutical substances that are to be administered perorally is reduced by efflux systems that are found in the intestinal epithelium, in particular P-glycoprotein transporters (P-gp- transporters).

In U.S. Pat. No. 6,028,054 A, the bioavailability of pharmaceutical substances is increased by the addition of so-called “bioenhancers” that reduce a gastrointestinal metabolization and/or a re-ejection by efflux systems. These “bioenhancers” are added to pharmaceutical preparations as additional components and consist of, e.g., two co-planar, aromatic rings with a positive charge.

In U.S. Pat. No. 6,121,234 A, the addition of ethereal oils to a pharmaceutical preparation that contains hydrophobic pharmaceutical substances is disclosed, whereby an inhibition of the enzymes of the cytochrome-P450-3A group and the efflux systems is based on the ethereal oils.

WO 99/11290 A1 and WO 01/003695 A1 disclose in each case an addition of bile acid propyl ester and vitamin C fatty acid ester, in particular vitamin C palmitate, to a pharmaceutical preparation that achieves an increase in bioavailability via an inhibition of enzymes of the cytochrome-P450-3A group.

In addition, in the finished pharmaceutical agent product Kaletra® with the active ingredient Lopinavir, which is responsible for a high enteral first-pass metabolism as well as outward transport by P-gp- transporters, Ritonavir is added as an adjuvant (Rote Liste [Red List], 2002), which at a concentration of ⅙ of its therapeutic dose reduces the enteral and probably also the hepatic metabolization and the outward transport by the P-gp- transporters of Lopinavir.

Drawbacks of this prior art consist in that, on the one hand, in each case at least one additional substance must be added to the pharmaceutical preparations to be able to achieve an inhibition of the respective systems, but according to the basic rules of the pharmaceutical formulation development, adjuvants are to be limited both qualitatively and quantitatively to a minimum. Another drawback consists in that the previously mentioned additives can have their own actions that can result in undesirable loads on the organism. In addition, other action-decisive metabolizations of pharmaceutical substances, such as, e.g., metabolizations via 17β-HSD, probably are not inhibited. 17β-HSD 2 has a high expression rate in intestinal and endometric tissue (MARTEL, C. et al.: J. Steroid Biochem. Molec. Biol. 1992, 41: 597-603) and metabolizes steroids, in particular those that in 17-position of the sterane skeleton have a secondary, optionally beta-position hydroxyl group in the corresponding ketone metabolites, such as, e.g., estradiol in estrone (cf. Zhu, B. T. et al., Carciogenesis, 1998, 19 (1): 1-27).

A method of solution to reduce the high oxidative metabolization rate of secondary, beta-position OH groups by the 17β-HSD 2 consists in achieving a steric stabilization of the sterane skeleton by, e.g., chemical substituents in 17α-position, cf., i.a., ethinylestradiol.

A galenical improvement of the inhibition of the metabolization of steroids with secondary, beta-position hydroxyl groups is disclosed in Price et al., Obstetrics & Gynecology 1997, 89, p. 340 ff by intestinal metabolization being avoided by sublingual administration.

From the above, it is now obvious that the problem of reducing the bioavailability of pharmaceutical substances that are substrates of intestinal enzymes, in particular 17β-HSD2 and/or CYP-3A4, and/or intestinal efflux systems, in particular P-gp- transporters, would still not be achieved in a satisfactory way in peroral dosage forms.

The object of the invention is therefore to develop a pharmaceutical preparation for peroral administration that, on the one hand, improves the solubility of, in particular, lipophilic pharmaceutical substances in especially spontaneous dilution with a hydrophilic medium, such as, e.g., the intestinal flow or purified water, and moreover, on the other hand, counteracts a reduction of the bioavailability of lipophilic and/or hydrophilic pharmaceutical substances by intestinal metabolization, in particular by 17β-HSD2 and/or CYP-3A4, or by an active outward transport from the intestinal cells by intestinal efflux systems, in particular P-gp- transporters (MDR1) or MRP2 proteins.

This object is achieved according to the invention by a preparation that contains at least one emulsifier, at least one auxiliary emulsifier and/or solvent as well as at least one lipid, characterized in that the mass ratio of emulsifier to auxiliary emulsifier and/or solvent (Smix) is 1:1 to 9:1 and the total lipid proportion is >0% (m/m), whereby this preparation at least partially inhibits at least one intestinal enzyme and/or at least one intestinal efflux system.

Pharmaceutical preparations according to the invention that inhibit intestinal enzymes and intestinal efflux systems are also referred to as enzyme-modulating-self-emulsifying-systems (EMSES).

In addition, the object is achieved by a use of a pharmaceutical preparation according to the invention for the production of a peroral pharmaceutical agent, whereby this preparation at least partially inhibits at least one intestinal enzyme and/or at least one intestinal efflux system.

In addition, the object according to the invention is achieved by a process for increasing the bioavailability of pharmaceutical substances that are to be administered perorally, whereby a pharmaceutical preparation according to the invention contains a pharmaceutical substance and is administered perorally.

An advantage of the pharmaceutical preparation according to the invention consists in that it improves the solubility of lipophilic and/or water-insoluble pharmaceutical substances compared to the prior art and at the same time at least partially reduces the metabolization of pharmaceutical substances by intestinal enzymes and/or the active outward transport by intestinal efflux systems.

Another advantage of an active ingredient-free pharmaceutical preparation according to the invention consists in that this preparation can be used as a carrier system for different pharmaceutical substances that have the previously mentioned profile, i.e., are lipophilic and/or are metabolized by intestinal enzymes and/or are transported back actively by intestinal efflux systems into the lumen of the bowels from the intestinal epithelium and thus can avoid time-consuming and costly pharmaceutical substance formulation developments.

Another advantage consists in that the pharmaceutical preparation according to the invention in addition is suitable to produce peroral dosage forms, especially capsules, advantageously gelatin capsules or tablets, based on a very small portion of a hydrophilic phase. In addition, this invention follows the basic rules of pharmaceutical formulation development, namely to limit adjuvants both qualitatively and quantitatively to a minimum.

Preferred embodiments are indicated in the subclaims.

A preferred embodiment of the invention consists in that the Smix is 3:1 to 9:1, advantageously 9:1, and the total lipid proportion is 10 to 50% (m/m).

An especially preferred embodiment of the invention consists in the fact that the emulsifier contains PEG-40-hydrogenated castor oil (Cremophor® RH40), PEG-35 castor oil (Cremophor®EL) or PEG-400-monoricinoleate (Estax®54), the auxiliary emulsifier or solvent glyceryl monocaprylate >80% (m/m) (Imwitor®308) or diethylene glycol monoethyl ether (Transcutol®P) and the lipid triglycerides, fatty oils or waxes.

Especially preferred triglycerides contain mid-chain triglycerides (MCT), e.g., Miglyol®812 (C₈-C₁₂ triglycerides); preferred fatty oils contain castor oil, olive oil, corn oil, soybean oil, sunflower seed oil, peanut oil, walnut oil or diestel oil, especially preferably castor oil; and preferred waxes contain ethyl oleate or isopropyl myristate.

It is also possible that the preparation according to the invention in addition contains at least one pharmaceutical substance.

Pharmaceutical substances that originate from the group of therapeutic agents, prophylactic agents or diagnostic agents and are formulated preferably with the preparation according to the invention are either lipophilic and/or water-insoluble, alternatively hydrophilic.

Preferred are lipophilic pharmaceutical substances that are formulated with the preparations according to the invention, and especially preferred are pharmaceutical substances that are substrates of at least one intestinal metabolizing enzyme and/or one intestinal efflux system. Here, in turn, those pharmaceutical substances are advantageously formulated that are substrates of the 17β-hydroxy-steroid-dehydrogenases or the cytochrome-P450-monooxygenases, especially advantageously from the group of cytochrome P 450 3A-monooxygenases (CYP3A4) or are substrates of a P-gp- transporter system. For the purposes of this application, substrate means at least one intestinal enzyme and/or an intestinal efflux system in that these substances, e.g., pharmaceutical substances or pharmaceutical adjuvants, interact with intestinal enzymes and are metabolized by the latter and/or are transported by interaction with intestinal efflux systems by the latter actively from the intestinal epithelium back into the lumen of the bowels.

As pharmaceutical substances, steroids represent a quite especially preferred type of embodiment of the preparation according to the invention, advantageously those that in 17-position of the sterane skeleton have a secondary, beta-position hydroxyl group and of these especially preferably estrogens, antiestrogens or androgens.

Pharmaceutical substances that represent substrates of 17β-HSD are cited below but are not limited to the list; they also contain salts and/or derivatives of these pharmaceutical substances:

11-α-hydroxynandrolone, 16-α-fluoroestradiol, 16-α-iodoestradiol, 16-β-fluoroestradiol, 2,4-dibromoestradiol, 2-chloroestradiol, 2-ethoxyestradiol, 2-fluoroestradiol, 2-hydroxyestriol, 2-methoxyestradiol, 2-methoxyestriol, 2-methoxymethylestradiol, 3-methoxyestriol, 4-bromoestradiol, 4-chloroestradiol, 4-fluoro-17β-estradiol, 4-hydroxyestradiol, 4-hydroxytestosterone, 4-methoxyestradiol, 5-β-androstan-17β-ol-3-one, 6-α-hydroxyestradiol, 3α, 17β-androstanediol, 3β, 17β-androstanediol androstanolone, androstenediol, bolanediol, bolazine, boldenone, clostebol, dacuronium bromide, 17-deacetylpancuronium, dideactetylvecuronium, vecuronium, 17β-dihydroequilin, 560 -dihydro-19-nortestosterone, 16α-bromo-7α-(N-butyl, N-methyl-undecanamide)-estra-1,3,5(10)-triene-3,17β-diol (EM-105), 16α-chloro-7α-(N-butyl, N-methyl-undecanamide)-estra-1,3,5(10)-triene-3,17β-diol (EM-139), 16α-iodo-7α-(N-butyl, N-methyl-undecanamide)-estra-1,3,5(10)-triene-3,17β-diol (EM-156), 16α-bromo-7α-(N-butyl, N-methyl-undecanamide)-estra-1,3,5(10)-triene-3,17β-diol (EM-220), epiestriol, epitiostanol, estetrol, estradiol, estradiol-3-glucuronide, estradiol-3-methylether, estradiol-3-sulfate, estradiol-3-benzoate, estradiol-3-hexahydrobenzoate, estramustine, estriol, estriol-3-glucuronide, estriol-3-sulfate, estriol-16-glucuronide, estrynamine, 17β-hydroxy-6-methylene-androsta-1,4-dien-3-one (FCE-25071), fulvestrant, 1-hydroxy-17β-estradiol, 2-hydroxy-17β-estradiol, 4-hydroxy-17β-estradiol, 6-hydroxy-17β-estradiol, 7-hydroxy-17β-estradiol, 15-hydroxy-17β-estradiol, 18-hydroxy-17β-estradiol, 7-(N-butyl-undecanamide)-3,17β-estra-1,3,5(10)-triene-3,17β-diol (ICI-160325), 7α-(N-butyl-undecanamide)-3,1713-estra-1,3,5(10)-triene-3,17β-diol (ICI-163964), estra-1,3,5(10)-triene-7β-(N-butyl)undecanamide-3,17β-diol (ICI-164275), 7α-(N-butyl, N-methyl-undecanamide)-estra-1,3,5(10)-triene-3,17β-diol (ICI-164384), inocoterone, estra-3-sulfamate-1,3,5(10),7-tetraene-3,17β-diol (J-1059), cycloprop[14S,15β]-3′,15-dihydro-estra-1,3,5(10)-triene-3,17β-diol (J-824), estra -1,3,5(10)-triene-3-sulfamate-17β-ol (J-995), mesterolone, methenolone, 16-methylene estradiol, metogest, nandrolone, nisterime, norclostebol, 3-octyloxy-5α-androst-3-en-17β-ol (octostanol), estradiol-17-phenylpropionate-estradiol-benzoate mixture (ORG-369-2), 7-ethyl-nandrolone (ORG-41640), 11β-chloromethyl-estra-3,17β-diol (ORG-4333), piperidinium-1-[(2β,3α,5α,16β,17β)-3,17-dihydroxy-2-(1-piperidinyl)androstan-16-yl]-1-methyl-bromide (ORG-7402), 17-deacetylrocuronium (ORG-9943), oxendolone, 11α-methoxy-7α-methyl-estra-3-17β-diol (PDC-7), quinestradol, 17β-hydroxy-7α-methyl-androst-5-en-3-one (RMI-12936), 11α-ethenyl-estra-3, 17β-diol (RU-39951), 11β-[4(dimethylamino)phenyl]-estra-3, 17β-diol (RU-43944), 7α-{4-[2-(dimethylamino)ethoxy]phenyl}-estra-3,17β-diol (RU-45144), 11β-{4-[(methylsulfonyl)oxy]phenyl}-estra-3,17β-diol (RU-48382), 11β-{4-[[5-[(4,4,5,5,5-pentafluoropentyl)sulfonyl]pentyl]oxy]phenyl}-estra-3,17β-diol (RU-58668), 17β-dihydroxy-9α-fluoro-11β-androsta-1,4-dien-3-one (SQ-27957), stenbolone, cycloprop[14R,15α]estra-3′,15-dihydro-3-methoxy-1,3,5(10)-trien-17β-ol (STS-593), cycloprop[14S, 15β]estra-3′,15-dihydro-3-methoxy-1,3,5(10)-trien-17α-ol (STS-651), testosterone, trestolone, trilostane, 13β-ethyl-8α-gona-1,3,5(10)-triene-3,16α,17β-triol (WY-5090), 13β-ethyl-8β-gona-1,3,5(10)-triene-3,16α,17β-triol, estra-2-{tricyclo[3.3.1.13,7]decyl}-1,3,5(10)-triene-3,17β-diol (ZYC-5), ent-estradiol, 8β-vinyl-estradiol, 11β-fluoro-7α-{5-[N-methyl-N-3-(4,4,5,5,5-pentafluoropentylthio)-propylamino]-pentyl}-estra-1,3,5(10)-triene-3,17β-diol (subsequently AE1), 11β-fluoro-7α-{5-[methyl-(7,7,8,8,9,9,10,10,10-nonafluorodecyl)amino]pentyl}estra-1,3,5(10)-triene-3,17β-diol (subsequently AE2), 17β-hydroxy-14α,15α-methylene-androst-4-en-3-one (WO 99/672275), 17β-hydroxy-7α-methyl-14α,15α-methylene-androst-4-en-3-one (WO 99/672275), 4-chloro-17β-hydroxy-14α,15α-methylene-androst-4-en-3-one (WO 01/42275), 4,17β-dihydroxy-14α,15α-methylene-androst-4-en-3-one (WO 01/42275), 17β-hydroxy-14α,15α-methylene-androsta-1,4-dien-3-one (WO 01/42275), 4-chloro-17β-hydroxy-14α,15α-methylene-androsta-1,4-dien-3-one (WO 01/42275), 4-chloro-17β-hydroxy-14α,15α-methylene-estr-4-en-3-one (WO 01/42274), 7β-hydroxy-7α-methyl-14α,15α-methylene-estr-4-en-3-one (WO 99/67276), 17β-hydroxy-14α,15α-methylene-estr-4-en-3-one (WO 99/67276), 4,17β-dihydroxy-14α,15α-methylene-estr-4-en-3-one (WO 01/42274), 17β-hydroxy-14α,15α-methylene-estra-4,9,11-trien-3-one (WO 01/42274), 3-ethyl-17β-hydroxy-14α,15α-methylene-gon-4-en-3-one (WO 01/42274), 17α-p-hydroxy-17α-homoandrosta-4,15-dien-3-one, and 1″-mesyl-17α-(trifluoromethyl)-1′H-pyrazol[4″,5′:2,3]androst-4-en-17β-ol.

Pharmaceutical substances and pharmaceutical substance groups that are lipophilic and/or represent substrates of cytochrome-P450 monooxygenases are cited below, but are not limited to this list; they also contain salts and/or derivatives of these pharmaceutical substances:

Aromatic hydrocarbons, arylamines, heterocyclic amines, caffeine, 1,3,7-trimethylxanthine, theophylline, odansertron, diethylnitrosamine, cyclophosphamide, R-methyl-phenytoin; antidiabetic agents—in particular glibenclamide, rosiglitazone and tolbutamide; non-steroidal anti-rheumatic agents (NSAR)—in particular diclofenac-Na and ibuprofen; coumarin, phenprocoumon, warfarin, sartanes, debrisoquin, sparteine, β-blockers, codeine; neuroleptic agents—in particular haloperiodol; phenothiazines, risperidone; selective serotonin reuptake inhibitors (SSRI)—in particular fluvoxamine; tricyclic antidepressants, nitrosamines, chloroxazone, dihydropyridines, triazolam, midazolam, astemizole, azole-antimycotic agents, cisapride; immunosuppressive agents—in particular cyclosporin, tacrolimus and sirolimus; calcium antagonists, macrolides; malaria agents—in particular halofantrine and mefloquine; pimozide; protease inhibitors—in particular saquinavir, ritonavir and Lopinavir, sildenafil; statins—in particular artorvastatin, fluvastatin, levostatin and simvastatin; steroids—estradiol, 11β-fluoro-17α-methyl-7α-{5-[methyl-(8,8,9,9,9-pentafluorononyl)amino]pentyl}estra-1,3,5(10)-triene-3,17β-diol (WO 03/045972), 11β-fluoro-7α-{5-[N-methyl-N-3-(4,4,5,5,5-pentafluoropentylthio)-propylamino]-pentyl}-estra-1,3,5(10)-triene-3,17β-diol (subsequently AE1), 11β-fluoro-7α-{5-[methyl-(7,7,8,8,9,9,10,10,10-nonafluorodecyl)amino]pentyl}estra-1,3,5(10)-triene-3,17β-diol (subsequently AE2), tamoxifen, and terfenadine.

Pharmaceutical substances and pharmaceutical substance groups that represent substrates of P-gp- transporters are cited below but are not limited to the list; they also contain salts and/or derivatives of these pharmaceutical substances:

Aldosterone, amidodarone, azipodines, bepridil, bisanthrene, catharanthine, cefazolin, cefoperazone, cefotetan, cefaranthine, chinchona alkaloids, chloropromazine, cisplatin, clozapine, cyclosporin, dexamethasone, dexniguldipine, dibucaine, digoxin, diltiazem, dipyridamole, domperidone, demetin, cis-flupenthixol, fluphenazine, flunitrazepam, gallopamil, haloperidol, hydrocortisone, ivermectin, loperamide, methadone, methotrexate, mitoxantrone, monesin, morphine, morphine 6-glucoronide, nicardipine, odansertron, perphenazine, phenoxazine, phenytoin, prazosin, progesterone, talinolol, tamoxifen, terfenadine, topotecan, trifluperazine, triflupromazine, valinomycin, verapamil, vindoline, yohimbine, ritonavir, Lopinavir, L-thyroxine, 11β-fluoro-17α-methyl-7α-{5-[methyl-(8,8,9,9,9-pentafluorononyl)amino]pentyl }estra-1,3,5(1 0)-triene-3,17β-diol (WO 03/045972), 11β-fluoro-7α-{5-[N-methyl-N-3-(4,4,5,5,5-pentafluoropentylthio) -propylamino]-pentyl}-estra-1,3,5(10)-triene-3,17β-diol (subsequently AE1), and 11β-fluoro-7α-{5-[methyl-(7,7,8,8,9,9,10,10,10-nonafluorodecyl)amino]pentyl}estra-1,3,5(10)-triene-3,17β-diol (subsequently AE2).

A preferred embodiment of the use of the preparations according to the invention consists in that P-gp- transporters are at least partially inhibited by the use of enzymes from the group of cytochrome-monooxygenases, preferably from the group of cytochrome-P450-3A-monooxygenases, or 17β-hydroxy-steroid dehydrogenases and/or as an intestinal efflux system. The capability of the pharmaceutical preparation according to the invention to increase the solubility of lipophilic phases, such as triglycerides, fatty oils or waxes, i.e., to make them readily water-dilutable, is shown by means of pseudotemary phase diagrams (cf. Example 3; FIGS. 1a, 1b). In this case, active ingredient-free preparations according to the invention that contain emulsifiers, auxiliary emulsifiers and/or solvents and lipids are diluted in steps with a hydrophilic medium, e.g., water. As evaluation criteria, clear/cloudy and single-phase/multi-phase apply. Clear to opalescent, single-phase systems were made visible in the pseudotemary phase diagrams (cf. FIGS. 1a and 1b) based on their occurrence at 25° C. or 37° C. It has now been shown that in contrast to pharmaceutical formulations from EP 670715 B1 in the case of a water-dilution in steps, no cloudiness develops in the dilution of the preparation according to the invention in a proportion of water over 70% (v/v+m), the so-called exhaust percentage, and that preparations according to the invention up to a proportion of water of 90% (v/v+m) have a transparent o/w dispersion.

The capability of the formulation according to the invention to be readily spontaneously water-dilutable and thus also to achieve a solubility of lipophilic pharmaceutical substances can be examined in the test on self-emulsifiability (cf. Example 4; FIGS. 2a-e). This test provides the processes, namely the spontaneous water dilution of formulations that are perorally administered in the stomach, again more true-to-life than the phase diagram since the preparation according to the invention is added to a hydrophilic phase that is introduced in excess. In this connection, preparations according to the invention are examined for their capability in spontaneous water dilution to form clear and homogeneous dispersions, i.e., their capability for self-emulsification. This test is evaluated, on the one hand, by a visual scale from 1 to 5 based on the rating system of Khoo et al., Int. J. of Pharmaceutics, 167 (1998) 155-164 (see Table 1) and, on the other hand, via the determination of the hydrodynamic particle diameter by means of photon correlation spectroscopy (PCS).

TABLE 1
Rating system of emulsions/dispersions after self-
emulsification (Khoo et al., 1998).

| Rating | Appearance of the System |
|---|---|
| 1 | Clear dispersion |
| 2 | Clear to opalescent dispersion |
| 3 | Whitish emulsion |
| 4 | Grayish emulsion |
| 5 | No self-emulsification, deposits on the |
| | surface of the water |

It has been found that formulations according to the invention that in the visual rating produce a clear and/or clear to opalescent dispersion and/or contain a particle size ≦200 nm, in particular ≦100 nm, are especially suitable, since the assumption can be made that the pharmaceutical substance is available in molecularly-dissolved form. Between active ingredient-free and active ingredient-containing formulations, no significant difference relative to particle size and the visual grading was found in the test on self-emulsification.

In addition, adjuvants that are preferably used for the pharmaceutical preparation according to the invention also have a capacity to at least partially inhibit intestinal enzymes or intestinal efflux systems. The suitability of pharmaceutical adjuvants for the preparation according to the invention generally increases with increasing inhibition. Such potentials can be determined with the test systems that are known to one skilled in the art.

The capability of the pharmaceutical formulation according to the invention to at least partially inhibit 17β-hydroxy-steroid-dehydrogenases is proven, on the one hand, by in-vitro 17β-HSD tests (cf. Example 5, FIG. 3). Pharmaceutical preparations according to the invention that contain steroids that have a secondary, beta-position hydroxyl group in 17-position of the sterane skeleton inhibit the 17β-HSD 2, so that less 17-keto-biotransformation product is produced. The inhibition potential is directly proportional to the mass proportion of the formulation according to the invention. With increasing concentration (0.0%, 0.003%, 0.01%, 0.03%, 0.1% and 0.3%) of a mixture, according to the invention, that consists of Cremophor®EL/Miglyol® 812/Transcutol®P (72T/20T/8T) in the test batch, the extent of the metabolization of 11β-fluoro-7α-{5-[methyl-(7,7,8,8,9,9,10,10,10-nonafluorodecyl)amino]pentyl}estra-1,3,5(10)-triene-3,17β-diol (AE2) to form 17-ketone is reduced after an incubation time of 30 minutes as follows (n=2): 20% and 23%→21% and 18%→14% and 15%→9% and 8%→5% and 6% −3% and 3% of the starting value of AE2, and it results in an increase of the amount of genuine AE2 after 30 minutes in the microsome suspension (n=2) 64% and 66%→70% and 71%→75% and 76%→89% and 83%→109% and 114%→106% and 109% of the starting value of AE2 (cf. Example 5; FIG. 3). It can be seen from this that formulations according to the invention are suitable to inhibit 17β-HSD 2.

Proof of the capability of the formulation according to the invention to increase the bioavailability of pharmaceutical substances, especially of steroids with a secondary, beta-position hydroxyl group in 17-position of the sterane skeleton, can be examined by means of an in-vivo test that is known to one skilled in the art. In this in-vivo test (cf. Example 6), the bioavailabilities of AE2 are determined in i.v. and p.o. administration. Since the active ingredient AE2 is very strongly lipophilic (log P=5.9), pharmaceutical preparations are used that in each case guarantee the solubility of the pharmaceutical substance in the respective formulation and thus already produce highly-developed systems. A 20% HP13CD solution that contains 2% AE2 and is administered both i.v. and p.o. is used as a reference. As a test formulation, the pharmaceutical preparation according to the invention that is presented in Example 2a) and that contains 2.5% AE2, Cremophor®EL and Transcutol®P, Smix 9:1, 20% (m/m) Miglyol®812 is administered p.o. In FIG. 4, it can be seen that the formulation of AE2 according to the invention achieves a bioavailability that is increased by 26% relative to this 20% HP-β-CD solution that is administered p.o. It can be seen from this that formulations according to the invention are suitable to increase the bioavailabilities of, in particular, steroids.

The capability of adjuvants to inhibit human cytochrome P450 isoenzymes is determined via a CYP test (cf. Example 7). In this connection, the inhibition of the enzymes is characterized via the concentration (IC₅₀, μg/ml) of the adjuvants, in which 50% of the respective isoenzymes are inhibited. It has been shown that the CYP-isoenzymes, in particular CYP3A4, are selectively inhibited by the adjuvants according to the invention. The adjuvants according to the invention on at least one CYP-isoenzyme have at least one IC₅₀<1000, advantageously an IC₅₀≦100, i.e., they have moderate activity, and especially advantageously an IC₅₀≦10, i.e., they have strong activity. In testing preparations according to the invention that are produced according to Example 1a) and/or 1d), it was found, surprisingly enough, that on isoenzymes CYP 3A4, CYP 2C9 and CYP 2C19, they have a lower IC₅₀ than the respective individual adjuvants and a moderate to strong activity (cf. Table 2).

TABLE 2
Inhibition Activities Relative to CYP-Isoenzymes of Adjuvants and Formulations

| | | IC50, μg/ml | | |
|---|---|---|---|---|
| | | CYP- | CYP- | CYP- |
| Name | Substance | 2C9 | 2C19 | 3A4 |
| Cremophor ® EL | Polyethylene glycol-35-castor oil | 2.1 | 10 | 16 |
| Cremophor ® RH40 | PEG-40-hydrogenated castor oil | 35 | 11 | 23 |
| Estax ® 54 | PEG-400-monoricinoleate | 1.6 | 0.7 | 3.8 |
| Miglyol ® 812 | C8-C12 triglycerides (MCT) | 350 | 100 | 245 |
| Refined castor oil | Refined castor oil (RIZ) | 1150 | >3000 | 930 |
| Transcutol ® P | Ethoxydiglycol | 1500 | 1400 | 755 |
| Ethyl oleate | Ethyl oleate (EO) | 195 | 325 | 117 |
| Imwitor ® 308 | Glycerol monocaprylate | 5.0 | 6.9 | 29 |
| Tween ® 80 | Polysorbate 80 | 2.9 | 8.4 | 7.3 |
| HPβCD (see | Hydroxypropyl-β-cyclodextrin | 1750 | 2900 | 1800 |
| Example 6) | | | | |
| Formulation | Cremophor ® EL: Transcutol ® P, 9:1 + | 21 | 6.4 | 11 |
| according to | 20% Miglyol ® 812 | | | |
| Example 1a | | | | |
| Formulation | Estax ® 54: Transcutol ® P, 9:1 + 20% | 1.4 | 2.1 | 4.1 |
| according to | Miglyol ® 812 | | | |
| Example 1d | | | | |

These results confirm that pharmaceutical preparations according to the invention inhibit intestinal enzymes, in particular 17β-HSD2 and cytochrome-isoenzymes, advantageously CYP3A4, and thus can result in an increase in the bioavailability of pharmaceutical substances.

The capability of adjuvants to inhibit P-gp- transporters is determined via a P-gp- transporter test (cf. Example 8). In this test, the activity to inhibit the transporters is characterized via ratio (R) that indicates the ratio of the fluorescence intensity of the test solution to the fluorescence intensity of the blank and is directly proportional to inhibiting the P-gp- transporters. Fluorescence intensity test solution corresponds to fluorescence intensity, measured at 485/535 nm (excitation or emission), of cells that contain test solution and calcein AM working solution. Fluorescence intensity blank corresponds to the fluorescence intensity, measured at 485/535 nm (excitation or emission) of cells that do not contain any test solution but do contain calcein AM-working working solution and thus are used as O-values.

The following maximum activities, R-values, relative to the inhibition of P-gp- transporters, transporters, are determined for the adjuvants below in Table 3:

TABLE 3
Inhibition Activities Relative to P-gp- Transporters of Adjuvants

| Adjuvants | Maximum R-Value |
|---|---|
| Cremophor ® EL | 2.42 |
| Estax ® 54 | 2.17 |
| Cremophor ® RH40 | 1.63 |
| Refined castor oil | 1.26 |
| PEG 400 | 1.26 |
| Imwitor ® 308 | 1.24 |
| Transcutol ® P | 1.18 |
| Miglyol ® 812 | 1.13 |

Adjuvants that have a ratio ≧1.18, advantageously ≧1.6 and especially advantageously ≧2.1, are preferably suitable to inhibit the active outward transport of pharmaceutical substances by intestinal efflux systems, especially by P-gp- transporters and to result in an increase of pharmaceutical substance bioavailability, whereby in this case the pharmaceutical substances must be substrates of the P-gp- transporter [R (AE1)=2.43; R (AE2)]. Those adjuvants are thus preferably suitable to be used as adjuvants for the pharmaceutical preparations according to the invention.

Based on their high water-dilutability and thus their good solubility of especially lipophilic pharmaceutical substances as well as their capability of inhibiting intestinal enzymes and/or the active outward transport by intestinal efflux systems, the pharmaceutical preparations according to the invention can be used as a technology platform for a wide variety of pharmaceutical substances, especially those mentioned previously. In Table 4, the formulations according to the invention that are preferably used for this purpose are indicated. In Table 5, possible active ingredient concentrations of selected pharmaceutical formulations according to the invention are indicated.

TABLE 4
Active-Ingredient-Free Formulations According to the Invention

| No. | Lipid | Emulsifier | Auxiliary Emulsifier | Smix |
|---|---|---|---|---|
| 1 | Mid-chain | POE-35-Glycerol- | Ethylene diglycol | 1:1 |
| | triglycerides | triricinoleate | monoethyl ether | |
| | (Miglyol ® 812) 10% (m/m) | (Cremophor ® EL) | (Transcutol ® P) | |
| 2 | Refined castor oil | POE-40-glycerol- | Glycerol | 3:1 |
| | 30% (m/m) | hydroxystearate | monocaprylate | |
| | | (Cremophor ® RH40) | (Imwitor ® 308) | |
| 3 | Mid-chain | POE-35-glycerol- | Ethylene diglycol | 9:1 |
| | triglycerides | triricinoleate | monoethyl ether | |
| | (Miglyol ® 812) 20% (m/m) | (Cremophor ® EL) | (Transcutol ® P) | |
| 4 | Mid-chain | POE-35-glycerol- | Glycerol | 3:1 |
| | triglycerides | triricinoleate | monocaprylate | |
| | (Miglyol ® 812) 30% (m/m) | (Cremophor ® EL) | (Imwitor ® 308) | |
| 5 | Ethyl oleate 40% | POE-35-glycerol- | Glycerol | 9:1 |
| | (m/m) | triricinoleate | monocaprylate | |
| | | (Cremophor ® EL) | (Imwitor ® 308) | |

TABLE 5
Active-Ingredient-Containing Formulations According to the Invention

| | | | | | WS (Active |
|---|---|---|---|---|---|
| | | | Auxiliary | | Ingredient) |
| No. | Lipid | Emulsifier | Emulsifier | Smix | Concentration |
| 1 | Mid-chain | POE-35-Glycerol- | Ethylene diglycol | 1:1 | Up to 500 mg |
| | triglycerides | triricinoleate | monoethyl ether | | of AE1 in 1 g |
| | (Miglyol ® 812) | (Cremophor ® EL) | (Transcutol ® P) | | of preconcentrate |
| | | | | | at |
| | | | | | 10% (m/m) |
| | | | | | lipid |
| 2 | Refined castor | PEG-400- | Ethylene diglycol | 9:1 | Up to 75 mg of |
| | oil | monoricinoleate | monoethyl ether | | AE2 in 1 g of |
| | | (Estax ® 54) | (Transcutol ® P) | | preconcentrate |
| | | | | | at 10% (m/m) |
| | | | | | lipid |
| 3 | Mid-chain | POE-35-Glycerol- | Ethylene diglycol | 9:1 | Up to 50 mg of |
| | triglycerides | triricinoleate | monoethyl ether | | AE2 in 1 g of |
| | (Miglyol ® 812) | (Cremophor ® EL) | (Transcutol ® P) | | preconcentrate |
| | | | | | at 20% (m/m) |
| | | | | | lipid |
| 4 | Mid-chain | POE-35-Glycerol- | Glycerol | 3:1 | Up to 37.5 mg |
| | triglycerides | triricinoleate | monocaprylate | | of E2 in 1 g of |
| | (Miglyol ® 812) | (Cremophor ® EL) | (Imwitor ® 308) | | preconcentrate |
| | | | | | at 10% (m/m) |
| | | | | | lipid |
| 5 | Ethyl oleate | POE-35-Glycerol- | Glycerol | 9:1 | Up to 50 mg of |
| | | triricinoleate | monocaprylate | | E2 in 1 g of |
| | | (Cremophor ® EL) | (Imwitor ® 308) | | preconcentrate |
| | | | | | at 10% (m/m) |
| | | | | | lipid |

The examples below represent preferred compositions of the invention without, however, limiting the invention to these examples.

### DESCRIPTION OF THE FIGURES

FIG. 1a:

Pseudotemary phase diagram of a mixture that consists of Cremophor®EL and Imwitor®308, Smix 9:1, Miglyol®812, titrated with water at 25° C. and 37° C. Leg designations run counterclockwise. Transparent single-phase areas at 25° C. and 37° C. are labeled in lengthwise stripes.

FIG. 1b:

Pseudotemary phase diagram of a mixture that consists of Cremophor®EL and Transcutol®P, Smix 3:1, Miglyol®812, titrated with water at 25° C. and 37° C. Leg designations run counterclockwise. Transparent, single-phase areas at 25° C. and 37° C. are labeled in lengthwise stripes and are filled in only at 37° C.

FIG. 2a:

Test for self-emulsification of a mixture that contains Cremophor®EL and Transcutol®P, Smix 9:1, 10 to 60% (m/m), Miglyol®812. Symbols of visual rating: ▭ and Δ; symbol of particle size determined by means of PCS (n=4, with standard deviation):

FIG. 2b:

Test for self-emulsification of a mixture that contains 2% (m/m) AE2, Cremophor®EL and Transcutol®P, Smix 9:1, 10 to 60% (mn/m), Miglyol®812. Symbols of visual rating: ▭ and Δ; symbol of particle size determined by means of PCS (double values):

FIG. 2c:

Test for self-emulsification of a mixture that contains 7.5% AE1, Cremophor®EL and Imwitor®308, Smix 3:1, 10 to 60% (m/m), Miglyol®812. Symbol of visual rating: ▭; symbol of particle size determined by means of PCS (double values):

FIG. 2d:

Test for self-emulsification of a mixture that contains 2% E2, Cremophor®EL and Transcutol®P, Smix 9:1, 10 to 60% (m/m), Miglyol®812. Symbol of visual rating: ▭; symbol of particle size determined by means of PCS (double values):

FIG. 2e:

Test for self-emulsification of a mixture that contains 2% AE2, Estax®54 and Transcutol®P, Smix 9:1, 10 to 60% (n/m) Miglyol®812. Symbol of visual rating: ▭; symbol of particle size determined by means of PCS (double values):

FIG. 3:

17β-HSD2 test on microsomes of intestinal origin. Metabolic stability of 0.3 μM of AE2 in an intestinal microsome suspension and production of 17-ketone metabolites after 30 minutes based on the concentration (m/v) of a formulation according to the invention that contains Cremophor®EL and Transcutol®P, Smix 9:1, 20% (m/m), Miglyol®812.

Y-axis: Proportion, in percent, of AE2 (symbol: dark bar) and ketone metabolite of AE2 (symbol: bar with lengthwise stripes)

X-axis: Proportion, in percent, of the formulation according to the invention.

FIG. 4:

Comparison of the serum concentrations of AE2 in female rats, filled symbols, and its 17-ketone metabolites, empty symbols, after i.v. and p.o. administration for the period of 0-24 hours, whereby in rats (R) 1 and 2:5 mg/kg of AE 2 is administered in a 20% HPβCD solution i.v.; in R 3:10 mg/kg of AE 2 is administered in a 20% HPPCD solution p.o.; and in R 5 and 6:10 mg/kg of AE 2 is administered in a preparation of Example 2a according to the invention. Y-axis: serum concentration in ng/ml; X-axis: time in hours, logarithmic scale.

### EXAMPLE 1

### Production of Pharmaceutical Preparations According to the Invention

a) 5 g of a pharmaceutical preparation according to the invention that contains Cremophor®EL, Transcutol®P and Miglyol®812 is produced. In this connection, all adjuvants are thoroughly shaken before use. 3.6 g of Cremophor®EL and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). This preparation is monitored visually for clarity and homogeneity, i.e., the beaker is held before a light source, alternatively before a black background, and the contents of the beaker do not have any optically observable cloudiness or floating particles, or any different phases. 1.0 g of Miglyol®812 is then added to the mixture and stirred for 5 minutes at 500 rpm in the above-mentioned magnetic stirrer. This pharmaceutical preparation, basic mixture, is then visually examined for clarity and homogeneity (see above).

b) 5 g of a pharmaceutical preparation according to the invention that contains Cremophor®EL, Imwitor®308 and Miglyol®812 is produced. For this purpose, all adjuvants must be thoroughly shaken before use. Imwitor®308 must be converted into a flowable form before use by heating to 40° C. in a heatable magnetic stirrer (Heidolph MR 3001 K). 3.6 g of Cremophor®EL and 400 mg of molten Imwitor®308 are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). 1.0 g of Miglyol®812 of the mixture is then added and stirred for 5 minutes at 500 rpm in the above-mentioned magnetic stirrer. This basic mixture is examined visually for clarity and homogeneity (see Example 1a).

c) 5 g of a pharmaceutical preparation according to the invention that contains Cremophor®RH40, Transcutol®P and Miglyol®812 is produced. Cremophor®RH40 must be converted into a flowable form before use by heating to 40° C. in a heatable magnetic stirrer (Heidolph MR 3001 K). Moreover, all adjuvants must be thoroughly shaken before use. 3.6 g of Cremophor®RH40 and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of Miglyol®812 is added to the mixture and stirred for 5 minutes at 500 rpm in the above-mentioned magnetic stirrer. This basic mixture is visually examined for clarity and homogeneity (see Example 1a).

d) 5 g of a pharmaceutical preparation according to the invention that contains Estax®54, Transcutol®P and Miglyol®812 is produced. For this purpose, all adjuvants must be thoroughly shaken before use. 3.6 g of Estax®54 and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of Miglyol®812 is added to the mixture and stirred for 5 minutes at 500 rpm in the above-mentioned magnetic stirrer. This basic mixture is visually examined for clarity and homogeneity (see Example 1a).

e) 5 g of a pharmaceutical preparation according to the invention that contains Estax®54, Imwitor®308 and Miglyol®812 is produced. For this purpose, all adjuvants must be thoroughly shaken before use. Imwitor®308 must be converted into a flowable form before use by heating to 40° C. in a heatable magnetic stirrer (Heidolph MR 3001 K). 3.6 g of Estax®54 and 400 mg of Imwitor®308 are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a).

Then, 1.0 g of Miglyol®812 is added to the mixture and stirred for 5 minutes at 500 rpm in the above-mentioned magnetic stirrer. This basic mixture is visually examined for clarity and homogeneity (see Example 1a).

f) 5 g of a pharmaceutical preparation according to the invention that contains Estax®54, Transcutol®P and refined castor oil is produced. For this purpose, all adjuvants must be thoroughly shaken before use. 3.6 g of Estax®54 and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of refined castor oil is added to the mixture and stirred for 5 minutes at 500 rpm in the above-mentioned magnetic stirrer. This basic mixture is visually examined for clarity and homogeneity (see Example 1a).

g) 5 g of a pharmaceutical preparation according to the invention that contains Estax®54, Transcutol®P and ethyl oleate is produced. For this purpose, all adjuvants must be thoroughly shaken before use. 3.6 g of Estax®54 and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of ethyl oleate is added to the mixture and stirred for 5 minutes at 500 rpm in the above-mentioned magnetic stirrer. This basic mixture is visually examined for clarity and homogeneity (see Example 1a).

h) 5 g of a pharmaceutical preparation according to the invention that contains Estax®54, Transcutol®P and polyethylene glycol 400 is produced. For this purpose, all adjuvants must be thoroughly shaken before use. 3.6 g of Estax®54 and 400 mg of polyethylene glycol 400 are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of Miglyol®812 is added to the mixture and stirred for 5 minutes at 500 rpm in the above-mentioned magnetic stirrer. This basic mixture is visually examined for clarity and homogeneity (see Example 1a).

i) 5 g of a pharmaceutical preparation according to the invention that contains Cremophor®RH40, Transcutol®P and refined castor oil is produced. Cremophor®RH40 must be converted into a flowable form before use by heating to 40° C. in a heatable magnetic stirrer (Heidolph MR 3001 K). Moreover, all adjuvants must be thoroughly shaken before use. 3.6 g of Cremophor®RH40 and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of refined castor oil is added to the mixture and stirred for 5 minutes at 500 rpm in the above-mentioned magnetic stirrer. This basic mixture is visually examined for clarity and homogeneity (see Example 1a).

j) 5 g of a pharmaceutical preparation according to the invention that contains Cremophor®RH40, Transcutol®P and ethyl oleate is produced. Cremophor®RH40 must be converted into a flowable form before use by heating to 40° C. in a heatable magnetic stirrer (Heidolph MR 3001 K). Moreover, all adjuvants must be thoroughly shaken before use. 3.6 g of Cremophor®RH40 and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of ethyl oleate is added to the mixture and stirred for 5 minutes at 500 rpm in the above-mentioned magnetic stirrer. This basic mixture is visually examined for clarity and homogeneity (see Example 1a).

k) 5 g of a pharmaceutical preparation according to the invention that contains Cremophor®RH40, Imwitor®308 and Miglyol®812 is produced. Imwitor®308 must be converted into a flowable form before use by heating to 40° C. in a heatable magnetic stirrer (Heidolph MR 3001 K). For this purpose, all adjuvants must be thoroughly shaken before use. 3.6 g of Estax®54 and 400 mg of Imwitor®308 are weighed on a “Genius” analytical scale (Sartorius, Göttingen) in a beaker and mixed for 5 minutes at 500 rpm in a magnetic stirrer (Heidolph MR 3001 K). The homogeneity and clarity are visually monitored (see Example 1a). Then, 1.0 g of Miglyol®812 is added to the mixture and stirred for 5 minutes at 500 rpm in the above-mentioned magnetic stirrer. This basic mixture is visually examined for clarity and homogeneity (see Example 1a).

l) 5 g of a pharmaceutical preparation according to the invention that contains Cremophor®EL, Transcutol®P and refined castor oil is produced. For this purpose, all adjuvants must be thoroughly shaken before use. 3.6 g of Cremophor®EL and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). This preparation is visually monitored for clarity and homogeneity, i.e., the beaker is held before a light source, alternatively before a black background, and the contents of the beaker do not have any optically observable cloudiness or floating particles, or any different phases. Then, 1.0 g of refined castor oil is added to the mixture and stirred for 5 minutes at 500 rpm in the above-mentioned magnetic stirrer. This pharmaceutical preparation, basic mixture, is then visually examined for clarity and homogeneity (see above).

m) 5 g of a pharmaceutical preparation according to the invention that contains Cremophor®EL, Transcutol®P and ethyl oleate is produced. For this purpose, all adjuvants must be thoroughly shaken before use. 3.6 g of Cremophor®EL and 400 mg of Transcutol®P are weighed on a “Genius” analytical scale (Sartorius, Göttingen) in a beaker and mixed for 5 minutes at 500 rpm in a magnetic stirrer (Heidolph MR 3001 K). This preparation is visually monitored for clarity and homogeneity, i.e., the beaker is held before a light source, alternatively before a black background, and the contents of the beaker do not have any optically observable cloudiness or floating particles, or any different phases. Then, 1.0 g of ethyl oleate is added to the mixture and stirred for 5 minutes at 500 rpm in the above-mentioned magnetic stirrer. This pharmaceutical preparation, basic mixture, is then visually examined for clarity and homogeneity (see above).

n) 5 g of a pharmaceutical preparation according to the invention that contains Cremophor®EL, Imwitor®308 and ethyl oleate is produced. Imwitor®308 must be converted into a flowable form before use by heating to 40° C. in a heatable magnetic stirrer (Heidolph MR 3001 K). For this purpose, all adjuvants must be thoroughly shaken before use. 3.6 g of Cremophor®EL and 400 mg of Imwitor®308 are weighed on a “Genius” analytical scale (Sartorius, Göttingen) in a beaker and mixed for 5 minutes at 500 rpm in a magnetic stirrer (Heidolph MR 3001 K). This preparation is visually monitored for clarity and homogeneity, i.e., the beaker is held before a light source, alternatively before a black background, and the contents of the beaker do not have any optically observable cloudiness or floating particles, or any different phases. Then, 1.0 g of ethyl oleate is added to the mixture and stirred for 5 minutes at 500 rpm in the above-mentioned magnetic stirrer. This pharmaceutical preparation, basic mixture, is visually examined for clarity and homogeneity (see above).

Examples 1a)-n) accordingly can be characterized with an Smix of 9:1 and a lipid content (m/m) in the basic mixture of 20%. Other combinations that consist of this adjuvant composition are produced, on the one hand, by a replacement of the lipid phase by the amphiphilic phase (Smix), such that the composition contains, e.g., 10% MCT (Miglyol®812) in the basic mixture, or by a replacement of the portion of emulsifier mixture (Smix) by the MCT that is used. The basic mixture then typically contained 30%, 40% or 50% of the total lipid proportion. In addition, other combinations, which are suitable as preparations according to the invention, can be produced in the same way by a change of the Smix to 3:1 or 1:1.

### EXAMPLE 2

### Production of Active Ingredient-Containing Pharmaceutical Preparations

a) 12.5 mg of 11β-fluoro-7α-{5-[methyl-(7,7,8,8,9,9,10,10,10-nonafluorodecyl)amino]pentyl}estra-1,3,5(10)-triene-3,17β-diol (AE 2) is admixed into 5 g of the pharmaceutical preparation from Example 1a) and stirred in the magnetic stirrer (Heidolph MR 3001K) until the active ingredient has dissolved to a clear form in the formulation (for examination of clarity, see Example 1a). To accelerate the pharmaceutical substance solubility in the basic solution, while being stirred in the above-mentioned magnetic stirrer, heat is input until about 40° C. is reached. After 24 hours, the clarity of the system is again examined (see Example 1a).

b) 10.0 mg of 1,3,5(10)-estratriene-3,17β-diol×1/2H₂O, named estradiol (E2) below, is admixed into 5 g of the pharmaceutical preparation of Example 11) and stirred in the magnetic stirrer (Heidolph MR 3001K) until the active ingredient has dissolved to a clear form in the formulation (for examination of clarity, see Example 1a). To accelerate the pharmaceutical substance solubility in the basic solution, while being stirred in the above-mentioned magnetic stirrer, heat is input until about 40° C. is reached. After 24 hours, the clarity of the system is again examined (see Example 1a).

c) 10.0 mg of AE 2 is admixed into 5 g of the pharmaceutical preparation of Example 1d) and stirred in the magnetic stirrer (Heidolph MR 3001K) until the active ingredient has dissolved to a clear form in the formulation (for examination of clarity, see Example 1a). To accelerate the pharmaceutical substance solubility in the basic solution, while being stirred in the above-mentioned magnetic stirrer, heat is input until about 40° C. is reached. After 24 hours, the clarity of the system is examined again (see Example 1a).

d) 37.5 mg of 11β-fluoro-7a-[5-methyl-{4,4,5,5,5-pentafluoropentyl-9sulfanyl]-propyl}amino)-pentyl]-estra-1,3,5(10)-triene-3,17β-diol (AE 1) is admixed into 5 g of the pharmaceutical preparation of Example 1b), whereby the Smix is 3:1, and stirred in the magnetic stirrer (Heidolph MR 3001 K) until the active ingredient has dissolved to a clear form in the formulation (for examination of clarity, see Example 1a). To accelerate the pharmaceutical substance solubility in the basic solution, while stirring is done in the above-mentioned magnetic stirrer, heat is input until about 40° C. is reached. After 24 hours, the clarity of the system is examined again (see Example 1a).

### EXAMPLE 3

### Test for Water Dilution In Steps: Pseudoternary Phase Diagram

In each case, 1.0 g of the basic mixtures according to the invention analogously to Example 1b), containing Cremophor®EL, Imwitor®308 (Smix 9:1) and Miglyol®812 [varying total lipid proportion of 0-100% (m/m)], and analogously to Example 1a), containing Cremophor®EL, Transcutol®P (Smix 3:1) and Miglyol®812 [varying total lipid proportion of 0-100% (m/m)], are added in each case in a magnetic stirring rod to in each case a 16 ml beveled test tube. Each of these preparations is homogeneously mixed with the aid of a mixer, MS 1 minishaker (IKA Company, Staufen), at the highest stage for 2-3 minutes. After the first visual rating at 25° C. (see Example 1a), in each case 10% (v/v+w) water is added in portions by means of an Eppendorf pipette to each basic mixture to produce this batch. The systems are thoroughly mixed after each titration step for about 10 seconds in the contact shaker. A tempered equilibration follows at 37° C. (Thermostat F20 NH of Julabo, Seelbach for a 15 1 water bath) at a stirring speed of about 270 rpm (Variomag® Telemodul 40S with 60 magnetic stirring positions). A visual rating at 37° C. (see Example 1a) takes place after 0.25-0.5 hour. Water is added up to a total water content of 90% (v/v+w) according to the method above.

In the course of these dilutions, semisolid and gel-like batches arise, and the latter are heated (water bath with 60° C.) and then homogenized.

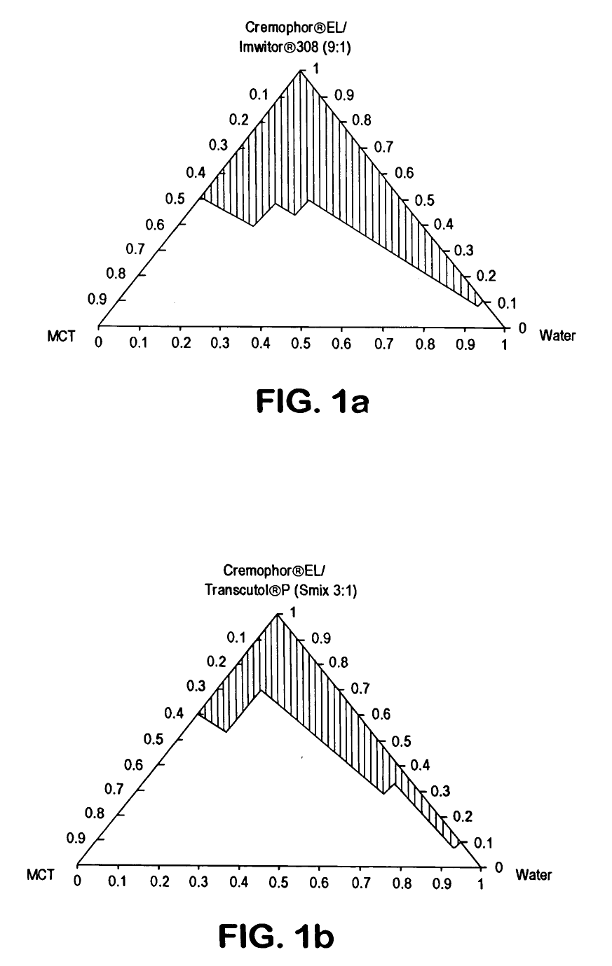

The thus obtained phase diagrams are depicted in FIGS. 1a and 1b and show that in particular basic mixtures according to the invention with a Smix of 9:1 up to a total lipid proportion of 50% (m/m) and basic mixtures according to the invention with a Smix of 3:1 up to a total lipid proportion of 30% (m/m) in the anhydrous basic mixture up to at least 90% (v/v+w) total water content have a clear and homogeneous emulsion and thus are suitable for the pharmaceutical preparations according to the invention.

### EXAMPLE 4

### Test for Self-Emulsifiability of Active-Ingredient-Free Pharmaceutical Preparations

a) In each case, 500 mg of the basic mixture, produced according to Example 1b), containing Cremophor®EL and Imwitor®308, Smix 9:1, and a respective total lipid proportion of 10, 20, 30, 40, 50 and 60% (m/m) Miglyol®812 are weighed on an analytical scale (“Genius,” Sartorius Company, Göttingen) in in each case a 1 ml disposable syringe. The filled syringe is added drop by drop to 250 ml of purelab® water that is heated to 37° C. and stirred in a release apparatus DT7R (ERWEKA, Heusen strain) with a blade-stirring attachment at 60 rpm. The release vessel is purified before the test with demineralized water and flushed at the end of the purification procedure with purelab® water.

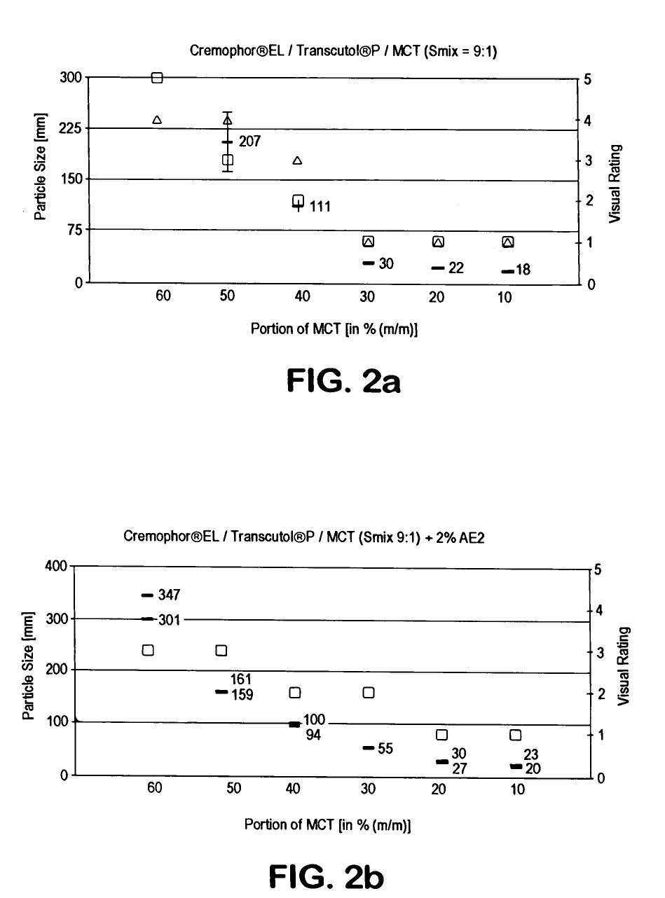

On the one hand, the dilutions are visually rated after 10 minutes, and, on the other hand, the particle size is determined by means of PCS (cf. FIG. 2a). The particle size measurement by PCS is carried out at a measuring temperature of 37° C. 1.46 was used as an angle of refraction of the dispersed phases. The viscosity value of these strongly diluted systems was allowed to remain in the basic setting of the device (viscosity value of water based on temperature). As can be seen in FIG. 2a), compositions with 10-40% (m/m) lipid in the preconcentrate are preferred, since they have clear to bluish shimmering dispersions (visual ratings of 1 and 2) with particle sizes of <200 nm. Especially preferred are compositions with 10-30% (m/m) lipid in the preconcentrate that have clear dispersions (visual ratings of 1) and particle sizes of <100 nm, since in this connection, it can be assumed that the active ingredient is available in a dissolved form.

The particle size was determined with a sample number n=4, such that the standard deviation according to the following formula

$$S = \sqrt{\frac{n \sum {x}^{2} - {( \sum x )}^{2}}{n ( n - 1 )}}$$

is incorporated into the figures. The open-triangle open symbols indicate the visual ratings of the different samples.

b) 500 mg of the active ingredient-containing basic mixture, produced analogously to Example 2d), containing 2% (m/m) AE2, Cremophor®EL and Imwitor®308, Smix 9:1, and a respective total lipid proportion of 10, 20, 30, 40, 50 and 60% (m/m) Miglyol®812 according to Example 4a) are tested for spontaneous water dilutability.

As can be seen in FIG. 2b), no significant difference in the visual rating, as also in the particle size, exists in FIG. 2a.

c) 500 mg of the active ingredient-containing basic mixture, produced analogously to Example 2d), containing 7.0% (m/m) AE1, Cremophor®EL and Imwitor®308, Smix of 3:1, and a respective total lipid proportion of 10, 20, 30, 40, 50 and 60% (m/m) Miglyol®812 according to Example 4a) are in each case tested for spontaneous water dilutability.

As can be seen in FIG. 2c), especially compositions with 10-50% (m/m) lipid in the preconcentrate are especially preferred, since they produce clear dispersions (visual ratings of 1) and particle sizes of <100 nm, and thus it can be assumed that the active ingredient is available in a dissolved form.

d) 500 mg of the active ingredient-containing basic mixture, produced analogously to Example 2b), containing 2.0% (m/m) E2, Cremophor®EL and Transcutol®P, Smix 9:1, and a respective total lipid proportion of 10, 20, 30, 40, 50 and 60% (m/m) castor oil (RIZ) according to Example 4a) in each case are tested for spontaneous water dilutability.

As can be seen in FIG. 2d), compositions with 10-40% (m/m) lipid in the preconcentrate are preferred, since they produce clear to bluish shimmering dispersions (visual ratings of 1 and 2) and particle sizes of <200 nm; especially preferred are compositions with 10-30% (m/m) lipid in the preconcentrate that produce clear dispersions (visual ratings of 1) and particle sizes of <100 nm, since in this connection, it can be assumed that the active ingredient is available in a dissolved form.

e) 500 mg of the active ingredient-containing basic mixture, produced analogously to Example 2c) and containing 2.0% (m/m) AE2, Estax®54 and Transcutol®P, Smix 9:1, and a respective total lipid proportion of 10, 20, 30, 40, 50 and 60% (m/m) Miglyole according to Example 4a) are tested in each case for spontaneous water dilutability.

As can be seen in FIG. 2e), compositions with 40 and 50% (m/m) lipid in the preconcentrate are preferred, since they produce clear to bluish shimmering dispersions (visual ratings: 2) and particle sizes of <200 nm. In these cases, it can be assumed that the active ingredient is available in a dissolved form.

### EXAMPLE 5

### Metabolic Stability, In Vitro 17β-HSD2 Test

A formulation according to the invention, produced analogously to Example 1a) and containing Cremophor®EL and Transcutol®P, Smix 9:1, 20% (m/m) Miglyol®812, is tested for its property to inhibit 17β-HSD2 in intestinal microsomes. 17β-HSD2 mediates the intestinal enzymatic dehydrogenation of an OH group in 17-position of the sterane skeleton to form a ketone group. An inhibition of the 17β-HSD2 is measured via a test substance AE2, which is the substrate of this enzyme, and is biotransformed into a 17-keto product. The ratio of the AE2 concentration and the 17-ketone-biotransformation product is determined at specific times (0, 10, 20, 30, 45 and 60 minutes) in each case for the starting-AE2 concentration in % (m/m).

For these tests, the following materials are used:

Na-Phosphate buffer: 100 mmol of Na₂HPO₄×2H₂O and 100 mmol of NaH₂PO₄×H₂O

Test substance solution of AE2: AE2 50 μmol in MeOH (in the test batch 0.3 μmol)

Formulation batches of the above-mentioned formulation according to the invention (% m/v): 0%, 0.00441%, 0.0147%, 0.0441%, 0.147% and 0.441% formulation in Na-phosphate buffer (in the test batch 0%, 0.003%, 0.01%, 0.03%, 0.01% and 0.3% formulation).

Cofactor solution: 2 ml of glucose-6-phosphate (160 mmol)/MgCl₂ (80 mmol)-mixture is added to 400 μl of a glucose-6-phosphate-dehydrogenase solution, then 15.6 mg of NADP and 13.4 mg of NAD are added.

Microsome solution: Intestinal microsomes (InVitro Technologies; protein content: 24 mg/ml; CYP450 content; 0.058 nmol/mg of protein) Thawed in a water bath at 37° C. (˜60 seconds) and diluted to a concentration of 5 mg/ml of protein with Na-phosphate buffer.

In each case, 170 μl/well of the formulation buffer and 5 μl/well of test substance solution of AE2 are introduced into the corresponding wells, whereby double values are set for each measuring time (0, 10, 20, 30, 45 and 60 minutes).

In each case, 250 μl of ice-cold MeOH is added at the 0-minute values. Immediately after, 25 μl of microsome solution and 50 μl of cofactor solution are added to all wells. The samples of the 0-minute values are stored without incubation at ˜−20° C. for about 24 hours. The other samples are incubated in each case for 10, 20, 30, 45 and 60 minutes at 37° C., and the dehydrogenation reaction is stopped after these times by the addition of 250 μl of ice-cold MeOH in each case. The samples are stored for about 24 hours before they are measured by HPLC at ˜−20° C. and they are centrifuged before HPLC analysis at 3000 rpm, whereby the supernatant is measured.

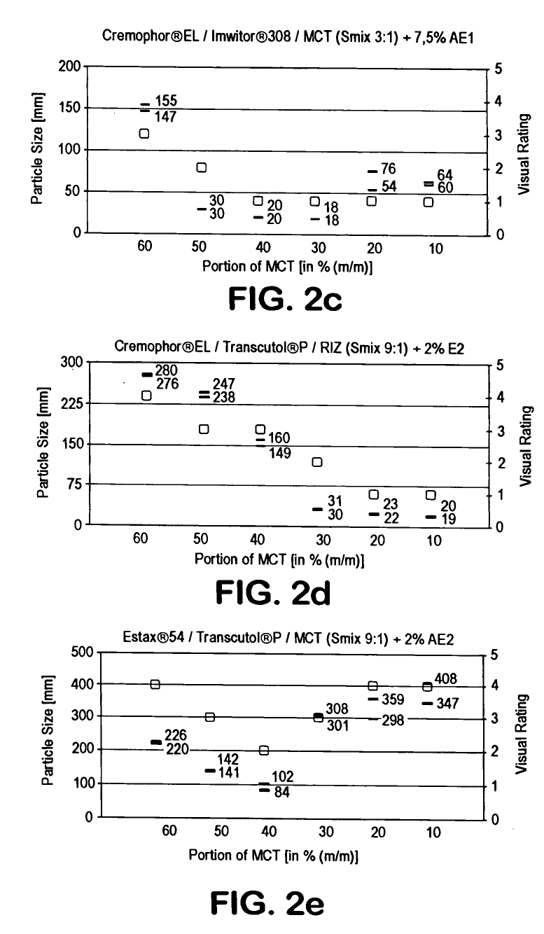

The concentrations of AE2 and 17 ketone product of AE2 that are measured by HPLC are presented in FIG. 3.

### EXAMPLE 6

In vivo i.v./p.o. Test for Inhibition of Intestinal 17β-HSD2 by Formulations According to the Invention in Rats

Animals used: Rats, female, 200-250 g, SchöWistar

HPβCD solution used: 20.0 g of hydroxypropyl-μ-cyclodextrin (HPβCD) is dissolved in 90 ml of water for injection purposes. 1.6 ml of a IN HCl solution is added to the cyclodextrin solution. Then, 2.0 g of AE2 is weighed in the aqueous cyclodextrin solution and dissolved at room temperature. 0.2 g of NaCl and 0.242 g of trometamol are weighed and dissolved in the active ingredient-containing cyclodextrin solution. The pH is set at 7.4 with 1N HCl. It is made up with water for injection purposes to a final volume of 100.0 ml and shaken. The solution is filtered with a 0.2 μmol membrane filter and autoclaved for 20 minutes at 121° C.

Formulation that is used according to the invention: Preparation produced analogously to Example 2a), containing 2% (m/m) AE2, Cremophor®EL and Transcutol®P, Smix 9:1, 20% (m/m) Miglyol®812

| | |
|---|---|
| Dose | i.v. 5 mg/kg of AE2 in 20% HPβCD solution |
| used: | p.o. 10 mg/kg of AE2 in 20% HPβCD solution or |
| | a formulation according to the invention |

The test extends over a period of 3 days.

| | |
|---|---|
| Day 1: | A catheterization of the jugular vein is performed on rats (R: 1, |
| | 2, 3, 5, 6) under Narkoren anesthesia. |
| Day 2: | R1 and R2: in each case 5 mg/kg of AE2-HPβCD solution is |
| | administered i.v. via the caudal vein; R3: 10 mg/kg of AE2- |
| | HPβCD solution is administered; and R 5 and 6: In each case |
| | 10 mg/kg of AE2-containing formulation according to the |
| | invention (mentioned above) is administered p.o. via the |
| | p.o.-administration needle. Blood samples are drawn at the |
| | specified times (i.v.: 5, |
| | 15, 30, 45 minutes; 1, 2, 4, 6, 8 and 24 hours; p.o.: 15, 30, 45 |
| | minutes; 1, 2, 4, 6, 8 and 24 hours) via the jugular catheter and |
| | worked up after sample working-up part 1. |
| Day 3: | Drawing of the 24 hour values from the vena cava, sample |
| | working-up parts 1 and 2 |

Sample Working-up:

| | |
|---|---|
| Part 1: | Serum recovery 30 minutes after the drawing of blood by |
| | centrifuging at 3000 g for 5 minutes. Then, 100 μl of the serum |
| | 1:5 is mixed with acetonitrile for precipitation. Sample storage |
| | until analysis by LC/MS/MS at ˜−20° C. (at least 24 hours). |
| Part 2: | Centrifuging of the precipitated serum (see part 1) at 5000 g |
| | for 5 minutes; pipetting-off of the aliquot for analysis by |
| | LC/MS/MS |

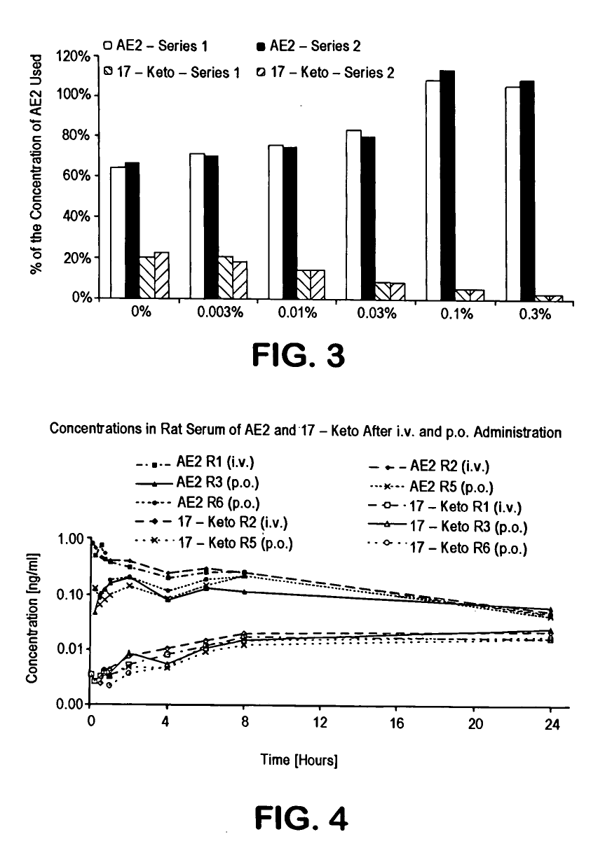

The pharmacokinetic parameters are calculated by means of the WinNonlin® program. The respective concentrations of AE2 and the metabolite are presented in FIG. 4.

### EXAMPLE 7

### Cytochrome P450 Inhibition Test, In Vitro CYP Test

Materials for These In-Vitro Tests:

96-hole plates, suitable for fluorescence measurements

Shaker/incubator for 37° C.

Plate-fluorescence reader (Fluostar)

| | |
|---|---|
| Incubation buffer: | Potassium-phosphate buffer, pH 7.4 (KP buffer) |
| Stop solution: | Acetonitrile/tris base 0.5 M, 80/20 (V/V) |

Solutions of test substances and positive controls in acetonitrile,
dilutions with incubation buffer.

| | |
|---|---|
| Test substances: | Cremophor ® EL, Cremophor ® RH40, |
| | Estax ® 54, Miglyol ® 812, refined castor oil, |
| | Transcutol ® P, ethyl oleate, Imwitor ® 308, |
| | Tween ® 80, 20% HPβCD solution (production |
| | described in Example 6), formulation according to |
| | the invention of Example 1a) and Example 1d) |

Stock solutions of the respective test substances or test formulations are produced in acetonitrile and diluted with incubation buffer (3 mg/ml in 2% acetonitrile in the batch); additional concentrations are produced by serial dilution. Concentration of the acetonitrile is at most 2% in all batches.

The incubations are set in 96-hole plate format with 200 μl of total volume in double values. The background batch controls the fluorescence of the batch without enzymes and formulations according to the invention, and the inherent fluorescence of the substances is determined in the buffer dilution. The preparation of the dilutions and the pipetting diagram corresponds to the original instructions of the manufacturer of the test kit (Gentest Corp., Woburn, Mass., USA).

The batches are started by adding the enzyme/substrate mixture, shaken for 30 or 45 minutes in an incubator at 37° C. and then interrupted with 50 μl of stop solution. The plates are briefly shaken, and the fluorescence intensity is measured in the plate-fluorescence reader at the wavelengths for excitation or emission.

The development of the fluorescent product forms the basis for the calculation. The comparison of the substrate conversion with or without the presence of the formulation according to the invention indicates an inhibition of the Cytochrome P450 isoenzyme. The IC₅₀ value is calculated from the concentration-dependent inhibition.

a) Inhibition of the Recombinant, Human Cytochrome
P450 3A4 Isoenzyme

| | | | |
|---|---|---|---|
| Incubation buffer: | KP buffer | 200 | mmol |
| Enzyme mixture: | Glucose-6-phosphate (G6P) | 0.4 | mmol |
| | MgCl₂ | 0.4 | mmol |
| | NADP+ | 8.1 | μmol |
| | G6Pdehydrogenase (G6PD) | 0.2 | IU/ml |
| | Recombinant human | 1 | pMol/batch |
| | CYP450-3A4, | | |
| Substrate: | 7-Benzyloxy- | 50 | μmol |
| | trifluoromethylcoumarin | | |
| | (7-BFC) | | |
| Positive Control: | Ketokonazole | 0.25-560 | nmol |
| Incubation period: | | 30 | minutes |
| Excitation/ | | 410/530 | nm |
| Emission | | | |
| wavelengths: | | | |

The results are presented in Table 2.

b) Inhibition of the Recombinant, Human
Cytochrome P450 2C9 Isoenzyme

| | | | |
|---|---|---|---|
| Incubation buffer: | KP buffer | 50 | mmol |
| Enzyme mixture: | Glucose-6-phosphate (G6P) | 0.4 | mmol |
| | MgCl₂ | 0.4 | mmol |
| | NADP+ | 8.1 | μmol |
| | G6Pdehydrogenase | 0.2 | IU/ml |
| | (G6PD) | | |
| | Recombinant human | 1.0 | pMol/batch |
| | CYP450-2C9, | | |
| Substrate: | 7-Methoxy-4- | 37.5 | μmol |
| | trifluoromethylcoumarin | | |
| | (7-MFC) | | |
| Positive Control: | Sulfaphenazole | 0.005-10 | μmol |
| Incubation period: | | 45 | minutes |
| Excitation/ | | 410/530 | nm |
| Emission | | | |
| wavelengths: | | | |

The results are presented in Table 2.

c) Inhibition of the Recombinant, Human Cytochrome
P450 2C19 Isoenzyme

| | | | |
|---|---|---|---|
| Incubation buffer: | KP buffer | 50 | mmol |
| Enzyme mixture: | Glucose-6-phosphate (G6P) | 0.4 | mmol |
| | MgCl₂ | 0.4 | mmol |
| | NADP+ | 8.1 | μmol |
| | G6Pdehydrogenase | 0.2 | IU/ml |
| | (G6PD) | | |
| | Recombinant human | 1.5 | pMol/batch |
| | CYP450-2C19, | | |
| Substrate: | 3-Cyano-7- | 25 | μmol |
| | ethoxycoumarin (CEC) | | |
| Positive Control: | Tranylcypromine | 0.045-100 | μmol |
| Incubation period: | | 30 | minutes |
| Excitation/ | | 410/460 | nm |
| Emission | | | |
| wavelengths: | | | |

The results are presented in Table 2.

### EXAMPLE 8

### Test For Inhibition of P-gp- Transporters; P-gp- Transporter Test

The following adjuvants are tested for their capability to inhibit human, intestinal P-gp- transporters: Cremophor®EL, Estax®54, Cremophor®RH40, refined castor oil, PEG 400, Imwitor®308, Transcutol®P, Miglyol®.

For this purpose, a suspension of human breast cancer cell lines, MATU-cell lines (Max-Dellbrück-Centrum (MDC)-Berlin-Buch; 40,000 cells/well/200 μl) is first added to a microtiter plate (Greiner black 96 plates, clear bottom, sterile). To ensure a stable and high expression of P-gp, the cells are cultivated over 3 days with adriamycin (ADR)-containing medium and then converted to ADR-free medium.

The culture medium contains 500 ml of RPMI (2.0 g/l of NaHCO₃; w/o L-glutamine, w/o phenol red, Article No.: F1275, Biochrom Company, Berlin), 5 ml of PenStrep® (10,000 U of penicillin, 10,000 μg/ml of streptomycin, Article No.: A2213, Biochrom Company, Berlin), 5 μl of L-glutamine (200 mmol, Article No.: K0283, Biochrom Company, Berlin), 50 μl of FCS (Article No.: S 0115, Biochrom Company, Berlin), and 50 μl of doxorubicin (1 μg/μl).

On day 4, the cells are washed for the test with incubation buffer and then equilibrated for about 10 minutes in incubation buffer. The incubation buffer, HEPES-carbonate buffer, pH 7.2, contains 128.1 mmol of NaCl, 5.4 mmol of KCl, 1.0 mmol of MgSO₄×7 H₂O, 1.8 mmol of CaCl₂×2 H₂O, 1.2 mmol of Na₂HPO₄×7 H₂O, 0.4 mmol of NaH₂PO₄×H₂O, 15.0 mmol of Hepes, 20.0 mmol of glucose, and 4.2 mmol of NaHCO₃. The cells that are differentiated in 96-microtiter plates are first washed 2× with the incubation buffer. The microtiter plate is now divided into zones. 180 μl of the respective test solution per well is added to a zone, and the entire microtiter plate is preincubated for 30 minutes at 37° C. The test solutions have concentrations of 0.3 to 33.3 μmol of test substances.

A 33.3 μmol concentrated Cremophor®EL test solution is produced from a 30 mmol stock solution of the adjuvant in DMSO, which is diluted 1:901 with incubation buffer to 33.3 μmol, and thus produces a concentration of 30 μmol in the batch, whereby the sum of the concentration of DMSO in the batch does not exceed 0.2% (v/v). Dilutions are produced analogously. Test solutions for the above-mentioned adjuvants are produced analogously.

Then, the addition of calcein AM working solution (20 μl/well) to all wells is carried out, i.e., with and without test solution. The plates are briefly carefully shaken and incubated for another 30 minutes at 37° C. The calcein AM working solution is diluted by diluting a 1 mmol calcein AM-stock solution in DMSO with incubation buffer to 10 μmol of calcein AM working solution. After incubation is completed, the fluorescence is measured in a plate-fluorescence reader (Fluostar®, B&L Systems, Maarssen) at 485/535 nm (excitation or emission).

The following ratio: ratio (R) of the fluorescence intensity of the test solution to the fluorescence intensity of the blank is determined.

The fluorescence intensity of the test solution corresponds to the fluorescence intensity, measured at 485/535 nm (excitation or emission), of the cells that contain the test solution and the calcein AM working solution.

The fluorescence intensity of the blank corresponds to the fluorescence intensity, measured at 485/535 nm (excitation or emission), of the cells that do not contain any test solution, but rather contain calcein AM working solution and thus are used as O-values.

The results are presented in Table 3.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius, and all parts and percentages are by weight, unless otherwise indicated.

The entire disclosures of all applications, patents and publications, cited herein and of corresponding U.S. Provisional Application Serial No. 60/416,920, filed Oct. 9, 2002, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Pharmaceutical preparation that contains at least one emulsifier, at least one auxiliary emulsifier and/or solvent as well as at least one lipid, characterized in that the mass ratio of emulsifier to auxiliary emulsifier and/or solvent (Smix) is 1:1 to 9:1 and the total lipid proportion is >0% (m/m), whereby this preparation at least partially inhibits at least one intestinal enzyme and/or at least one intestinal efflux system.

2. Pharmaceutical preparation according to claim 1, wherein the Smix is 3:1 to 9:1.

3. Pharmaceutical preparation according to claim 3, wherein the Smix is 9:1.

4. Pharmaceutical preparation according to claim 1, wherein the total lipid proportion is 10-50% (m/v).

5. Pharmaceutical preparation according to claim 1, whereby intestinal enzymes originate from the group of 17β-hydroxy-steroid-dehydrogenase or the cytochrome monooxygenases and intestinal efflux systems from the group of P-glycoproteins.

6. Pharmaceutical preparation according to claim 1, wherein the emulsifier contains PEG-40-hydrogenated castor oil (Cremophor®RH40), PEG-35 castor oil (Cremophor®EL) or PEG-400-monoricinoleate (Estax®54).

7. Pharmaceutical preparation according to claim 1, wherein the auxiliary emulsifier and/or the solvent contains glyceryl monocaprylate >80% (m/m) (Imwitor®308) or diethylene glycol monoethyl ether (Transcutol®P).

8. Pharmaceutical preparation according to claim 1, wherein the lipid contains triglycerides, fatty oils or waxes.

9. Pharmaceutical preparation according to claim 8, wherein the triglyceride contains mid-chain triglycerides (Miglyol®).

10. Pharmaceutical preparation according to claim 8, wherein the fatty oil contains castor oil, olive oil, corn oil, soybean oil, sunflower oil, peanut oil, walnut oil or diestel oil.

11. Pharmaceutical preparation according to claim 8, wherein the wax contains ethyl oleate or isopropyl myristate.

12. Pharmaceutical preparation according to claim 1, wherein the preparation contains in addition at least one pharmaceutical substance.

13. Pharmaceutical preparation according to claim 12, wherein the pharmaceutical substance is lipophilic and/or water-insoluble or hydrophilic.

14. Pharmaceutical preparation according to claim 1, wherein at least one pharmaceutical substance is a substrate of at least one intestinal enzyme and/or an intestinal efflux system.

15. Pharmaceutical preparation according to claim 14, wherein at least one intestinal enzyme originates from the group of 17β-hydroxy-steroid-dehydrogenases and/or cytochrome-monooxygenases.

16. Pharmaceutical preparation according to claim 15, wherein at least one intestinal enzyme is 17β-HSD 2 and/or originates from the group of cytochrome P 450 3A-monooxygenases.

17. Pharmaceutical preparation according to claim 14, wherein at least one intestinal efflux system originates from the group of P-gp- transporter systems.

18. Pharmaceutical preparation according to claim 1, wherein at least one pharmaceutical substance is a steroid.

19. Pharmaceutical preparation according to claim 18, wherein the steroid in 17-position of the sterane skeleton contains a secondary, beta-position hydroxyl group.

20. Pharmaceutical preparation according to claim 1, wherein the steroid is an estrogen, an antiestrogen or an androgen.

21. Pharmaceutical preparation according to claim 1, wherein the steroid 11-α-hydroxynandrolone, 16-α-fluoroestradiol, 16-α-iodoestradiol, 16-β-fluoroestradiol, 2,4-dibromoestradiol, 2-chloroestradiol, 2-ethoxyestradiol, 2-fluoroestradiol, 2-hydroxyestriol, 2-methoxyestradiol, 2-methoxyestriol, 2-methoxymethylestradiol, 3-methoxyestriol, 4-bromoestradiol, 4-chloroestradiol, 4-fluoro-17β-estradiol, 4-hydroxyestradiol, 4-hydroxytestosterone, 4-methoxyestradiol, 5-β-androstan-17β-ol-3-one, 6-α-hydroxyestradiol, 3α, 17β-androstanediol, 3β,17β-androstanediol, androstanolone, androstenediol, bolanediol, bolazine, boldenone, clostebol, dacuronium bromide, 17-deacetylpancuronium, dideactetylvecuronium, vecuronium, 17β-dihydroequilin, 5α-dihydro-19-nortestosterone, 16α-bromo-7α-(N-butyl, N-methyl-undecanamide)-estra -1,3,5(10)-triene-3,17β-diol, 16α-chloro-7α-(N-butyl, N-methyl -undecanamide)-estra-1,3,5(10)-triene-3,17β-diol, 16α-iodo-7α-(N-butyl, N -methyl-undecanamide)-estra-1,3,5(10)-triene-3,17β-diol, 16α-bromo-7α-(N -butyl, N-methyl-undecanamide)-estra-1,3,5(10)-triene-3,17β-diol, epiestriol, epitiostanol, estetrol, estradiol, estradiol-3-glucuronide, estradiol-3-methylether, estradiol-3-sulfate, estradiol-3-benzoate, estradiol-3-hexahydrobenzoate, estramustine, estriol, estriol-3-glucuronide, estriol-3-sulfate, estriol-16-glucuronide, estrynamine, 17β-hydroxy-6-methylene -androsta-1,4-dien-3-one, fulvestrant, 1-hydroxy-17β-estradiol, 2-hydroxy-17β-estradiol, 4-hydroxy-17β-estradiol, 6-hydroxy-17β-estradiol, 7-hydroxy-17β-estradiol, 15-hydroxy-17β-estradiol, 18-hydroxy-17β-estradiol, 7-(N-butyl -undecanamide)-3,17β-estra-1,3,5(10)-triene-3,17β-diol, 7α-(N-butyl -undecanamide)-3,17β-estra-1,3,5(10)-triene-3,17β-diol, estra-1,3,5(10)-triene -7β-(N-butyl)undecanamide-3,17β-diol, 7α-(N-butyl, N-methyl-undecanarnide)-estra-1,3,5(10)-triene-3,17β-diol, inocoterone, estra-3-sulfamate-1,3,5(10),7-tetraene-3,17β-diol, cycloprop[14S,15β]-3′,15-dihydro -estra-1,3,5(10)-triene-3,17β-diol, estra-1,3,5(10)-triene-3-sulfamate-17β-ol, mesterolone, methenolone, 16-methyleneestradiol, metogest, nandrolone, nisterime, norclostebol, 3-octyloxy-5α-androst-3-en-17β-ol, estradiol-17-phenylpropionate-estradiol-benzoate mixture, 7-ethyl-nandrolone, 11β-chloromethyl-estra-3,17β-diol, piperidinium-1-[(2β,3α,5α,16β,17β)-3,17-dihydroxy-2-(1-piperidinyl)androstan-16-yl]-1-methyl-bromide, 17-deacetylrocuronium, oxendolone, 11α-methoxy-7α-methyl-estra-3-17β-diol, quinestradol, 17β-hydroxy-7α-methyl-androst-5-en-3-one, 11α-ethenyl-estra-3, 17β-diol, 11β-[4(dimethylamino)phenyl]-estra-3, 17β-diol, 7α-{4-[2-(dimethylamino)ethoxy]phenyl}-estra-3,17β-diol, 11β-{4-[(methylsulfonyl)oxy]phenyl}-estra-3,17β-diol, 11β-{4-[[5-[(4,4,5,5,5-pentafluoropentyl)sulfonyl]pentyl]oxy]phenyl}-estra-3,17β-diol, 17β-dihydroxy-9α-fluoro-11β-androsta-1,4-dien-3-one, stenbolone, cycloprop[14R,15α]estra-3′,15-dihydro-3-methoxy-1,3,5(10)-trien-17β-ol, cycloprop[14S,15β]estra-3′,15-dihydro-3-methoxy-1,3,5(10)-trien-17β-ol, testosterone, trestolone, trilostane, 13β-ethyl-8α-gona-1,3,5(10)-triene -3,16α,17β-triol, 13β-ethyl-8β-gona-1,3,5(10)-triene-3,16α,17β-triol, estra-2-{tricyclo[3.3.1.13,7]decyl}-1,3,5(10)-triene-3,17β-diol, ent-estradiol, 8β-vinyl-estradiol, 11β-fluoro-7α-{5-[N-methyl-N-3-(4,4,5,5,5-pentafluoropentylthio)-propylamino]pentyl}-estra-1,3,5(10)-triene-3,17β-diol, 11β-fluoro-7α-{5-[methyl-(7,7,8,8,9,9,10,10,10-nonafluorodecyl)amino]pentyl}estra-1,3,5(10) -triene-3,17β-diol, 11β-fluoro-17α-methyl-7α-{5-[methyl-(8,8,9,9,9-pentafluorononyl)amino]-pentyl}estra-1,3,5(10)-triene-3,17β-diol, 17β-hydroxy-14α,15α-methylene-androst-4-en-3-one, 17β-hydroxy-7α-methyl -14α,15α-methylene-androst-4-en-3-one, 4-chloro-17β-hydroxy-14α,15α-methylene-androst-4-en-3-one, 4,17β-dihydroxy-14α,15α-methylene-androst -4-en-3-one, 17β-hydroxy-14α,15α-methylene-androsta-1,4-dien-3-one, 4-chloro-17β-hydroxy-14α,15α-methylene-androsta-1,4-dien-3-one, 4-chloro -17β-hydroxy-14α,15α-methylene-estr-4-en-3-one, 17β-hydroxy-7α-methyl -14α,15α-methylene-estr-4-en-3-one, 17β-hydroxy-14α,15α-methylene-estr-4-en-3-one, 4,17β-dihydroxy-14α,15α-methylene-estr-4-en-3-one, 17β-hydroxy -14α,15α-methylene-estra-4,9,11-trien-3-one, 3-ethyl-17β-hydroxy-14α,15α-methylene-gon-4-en-3-one, 17a-β-hydroxy-17a-homoandrosta-4,15-dien-3-one, 1″-mesyl-17α-(trifluoromethyl)-1′H-pyrazol[4″,5′:2,3]androst-4-en-17β-ol.

22. Use of a pharmaceutical preparation according to claim 1 for the production of a peroral pharmaceutical agent for inhibiting at least one intestinal enzyme and/or at least one intestinal efflux system.

23. Use of a pharmaceutical preparation according to claim 22, wherein at least one intestinal enxyme originates from the group of 17β-hydroxy -steroid-dehydrogenases and/or cytochrome-P450-monooxygenases.

24. Use of a pharmaceutical preparation according to claim 23, wherein at least one intestinal enzyme is 17β-HSD 2 and/or originates from the group of cytochrome-P450-3A-monooxygenases.

25. Use of a pharmaceutical preparation according to claim 22, wherein at least one intesinal efflux system is a P-gp-transporter.

26. Use of a pharmaceutical preparation according to claim 1, wherein the pharmaceutical agent comes from the group of therapeutic agents, prophylactic agents or diagnostic agents.

27. Process for increasing the bioavailability of pharmaceutical substance that are to be administered perorally, wherein a pharmaceutical preparation contains a pharmaceutical substance and is administered perorally according to claim 1.
